(19) United States
(12) Patent Application Publication
(10) Pub. No.: US 2011/0245786 A1
(43) Pub. Date: Oct. 6, 2011

(54) Wound Irrigation Device

(76) Inventor: Janet Hulse, Campbell Hall, NY (US)

(21) Appl. No.: 12/984,450

(22) Filed: Jan. 4, 2011

Related U.S. Application Data
(60) Provisional application No. 61/292,041, filed on Jan. 4, 2010.

(51) Int. Cl. A61M 5/315

(52) U.S. Cl. 604/311

(57) ABSTRACT

A wound irrigation device capable of discharging a constant, high pressure stream of a cleansing solution is contemplated. The device comprises a syringe-like device having a two piece construction. The syringe-like device includes a barrel for securely containing a predetermined volume of cleansing solution. The barrel includes an open end for insertion of a plunger to force the volume of cleansing solution out through an outlet on the opposite end of the barrel.

### BACKGROUND OF THE INVENTION

1. Field of the Invention

The present invention relates generally to medical devices, and more particularly, to a wound irrigation device suitable for cleaning wounds.

2. Discussion of the Related Art

High-pressure irrigation is a commonly known method of emergency wound care utilized for cleansing wounds. Successful wound irrigation is highly dependent upon a constant high pressure flow of saline or other cleansing agent such as sterile water. Wound irrigation is performed to expel or otherwise remove foreign bodies from the wound in order to prevent infection and remove debris.

In practice, a predetermined amount of solution is provided in a sterile container. The caregiver then utilizes a syringe to draw a desired amount of the solution therein. The syringe is then fitted with a catheter and positioned proximate to the wound to be cleansed, and the caregiver provides pressure to the plunger of the syringe to eject the solution from the syringe through the catheter and into the wound area. As is generally understood, the pressure at which the fluid irrigates the wound is dependent upon the rate at which the plunger of the syringe is depressed by the caregiver. Understandably, it is relatively difficult to maintain a constant and/or adequately high pressure of solution irrigation when pressure must be manually applied by the caregiver using known devices. Thus, the efficacy of known wound irrigation devices is highly dependent upon the caregiver performing the irrigation. Further, to properly cleanse the wound, a volume of solution greater than that typically contained by a syringe is required. Thus, the caregiver must continually refill the syringe, which requires disassembly of the catheter from the syringe. This results in a relatively time consuming process that increases the risk of the patient developing an infection.

In light of the foregoing, alternative devices for wound irrigation have been developed to attempt to overcome the disadvantages persistent in prior devices. However, these alternative designs are not without their own disadvantages. For instance, U.S. Pat. No. 5,133,701 to Han discloses a disposable pressure wound irrigation device. The device of the '701 patent provides a reservoir of cleansing solution such as, for example, saline provided in communication with a pressure chamber for providing force to the reservoir such that a stream of saline emitted by the device is expelled at a substantially constant pressure. The device of the '701 patent, however, is relatively cumbersome as compared to prior art devices. In particular, the device of the '701 patent requires the caregiver to use both hands to properly irrigate the wound. Further, the device itself is relatively complicated and includes a number of parts susceptible to mechanical error. For instance, the device requires a quantity of compressed gas in order to emit the cleansing solution at a preferred pressure. In addition, the device of Han is only capable of handling 250 ml of cleansing solution, which is understood in the art as an insufficient volume for completely irrigating most wounds. Thus, use of the Han device requires the use of more than one device to properly irrigate a wound. Finally, due to the relatively complicated nature of the device of the '701 patent, the device is relatively expensive to produce, which is particularly undesirable for a single-use medical device.

Thus, it is an object of the wound irrigation device of the present invention to provide a relatively simple wound irrigation device that does not suffer from the disadvantages suffered by known prior art devices. In particular, the wound irrigation device according to the present invention is adapted for application of a constant, high-pressure stream of cleansing solution to a wound area. Further, the device is relatively simple and inexpensive to produce. Finally, the device is relatively simple to operate and therefore it may be readily operated by a number of different caregivers including highly trained professionals such as doctors and registered nurses, as well as untrained caregivers.

### SUMMARY OF THE INVENTION

The wound irrigation device according to the present invention is adapted to deliver a stream of sterile solution at a constant, relatively high pressure. The irrigation device is generally syringe-like, comprising a relatively rigid cylindrical barrel and a plunger assembly. The two-part device may be constructed from a relatively durable plastic or the like. The barrel defines a cavity therein for receiving and securely storing a predetermined quantity of solution. The barrel and plunger assembly are adapted and configured to cooperate with one another such that the barrel receives the plunger therethrough. The plunger may be substantially hollow and includes a first end and a second end. Both the first and second ends of the plunger may comprise generally solid, flat surfaces. The plunger is configured to be pushed from one of first end and second end through the barrel so as to rupture a thin seal provided between the solution housed within the barrel and an outlet tube of the barrel. The liquid is then pushed out the outlet tube from a top of the barrel. The outlet tubing includes a tip at an end thereof opposite the lower end of the barrel. The tip includes a protective cap that is removed prior to use for maintaining the sterility of the solution within. A splash guard may be additionally provided for protecting caregivers using the device of the present invention. The splash guard may be constructed from plastic or a similar such material and may be integrally formed into the barrel such that it houses the tip of the device and extends outward and upwardly from the top edge of the barrel.

### BRIEF DESCRIPTION OF THE DRAWINGS

Preferred exemplary embodiments of the invention are illustrated in the accompanying drawings, in which like reference numerals represent like parts throughout, and in which:

FIG. 1 is a front elevation view of the wound irrigation device of the present invention;

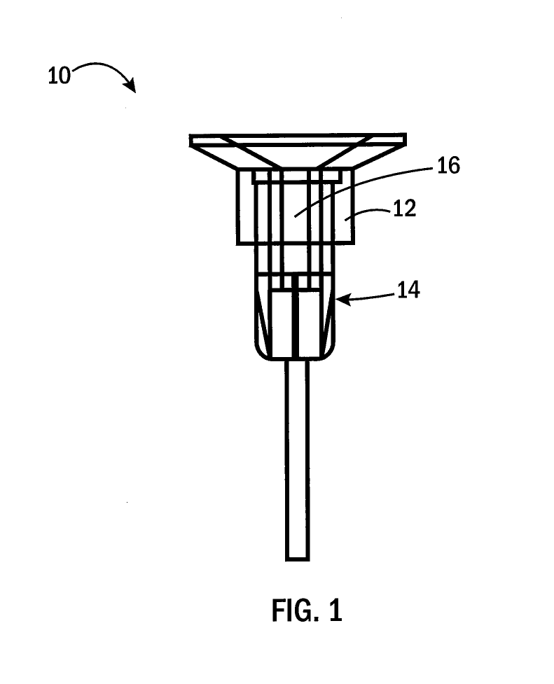

FIG. 2 is a partial front elevation view of the wound irrigation device of FIG. 1, illustrating a barrel thereof;

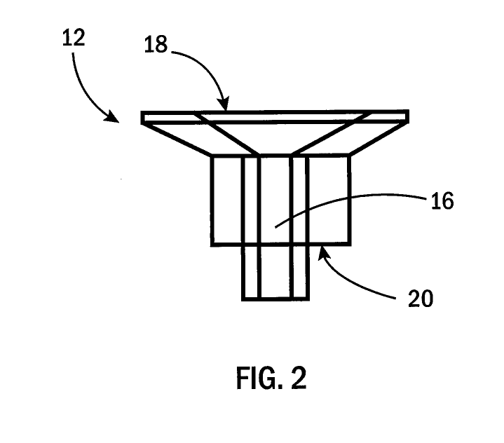

FIG. 3 is a partial front elevation view of the wound irrigation device of FIG. 1, illustrating a plunger thereof;

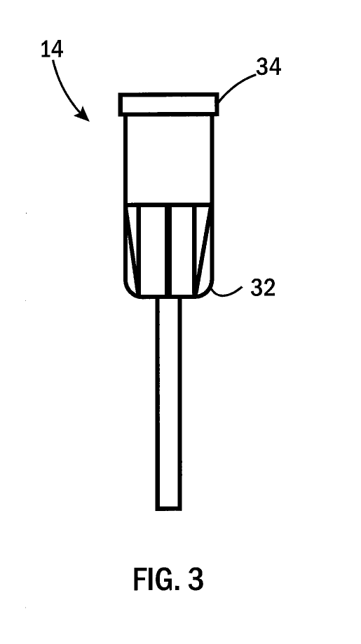

FIG. 4 is a cross-sectional view of the barrel of the wound irrigation device of FIG. 1; and

FIG. 5 is a cross-sectional view of the plunger of the wound irrigation device of FIG. 1.

### DETAILED DESCRIPTION OF THE PREFERRED EMBODIMENTS

Referring now to the drawings, the wound irrigation device 10 has a two-part construction including a barrel 12 and a plunger 14. Barrel 12 may be constructed from a relatively durable, rigid material such as plastic. In one embodiment, barrel 12 is generally cylindrical in shape, however other shapes are within the scope of the present invention. Barrel 12 is substantially hollow and defines an interior 16 for receiving and securely storing a predetermined amount of a cleansing solution for use in irrigating a patient's wound of particulate matter that may carry germs and bacteria capable of causing infection. The cleansing solution may be a standard saline solution as is generally understood in the art. Barrel 12 is preferably sized so as to store approximately 500 cubic centimeters (ccs) of cleansing solution, which is currently considered ideal for would cleansing purposes. However, barrel 12 may be sized to accommodate a wide number of different volumes as may be desired. Understandably, it may be advantageous for a particular hospital, clinic, or other such location to have a number of different sized devices 10 for accommodating wounds of varying size.

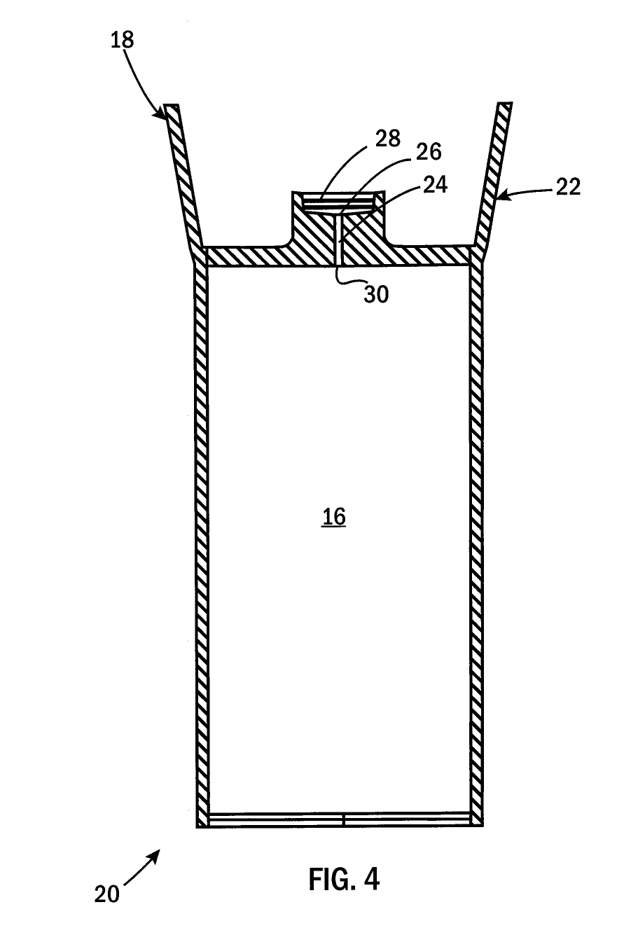

Referring now to FIG. 4 in particular, barrel 12 includes a top 18 and a bottom 20. Top 18 of barrel 12 is substantially solid and is generally adapted for emission of the cleansing solution therefrom while bottom 20 defines an aperture for receiving plunger 14 as will be described herein. Top 18 includes a splash guard 22. Splash guard 22 may be integrally formed with the barrel 12, or alternatively, splash guard 22 may comprise a separate component that may be selectively secured to top 18 of barrel 12 as desired. Splash guard 22 comprises a frustoconical shape and extends upwardly and outwardly from top 18 to define an interior thereof. An outlet 24 is integrally formed in top 18 of barrel 12 and is in communication with interior cavity 16 thereof. Outlet 24 includes a tip 26. Tip 26 is preferably an 18-gauge tip, although other sizes are within the scope of the present invention. A protective cap 28 is provided over tip 26 to maintain the sterility of tip 26. Cap 28 is configured to be selectively removable for allowing the discharge of the cleansing solution of barrel 12 through outlet 24. A seal 30 is provided between interior cavity 16 and outlet 24. Seal 30 is selectively rupturable under the application of pressure from the plunger 14 as will be discussed herein.

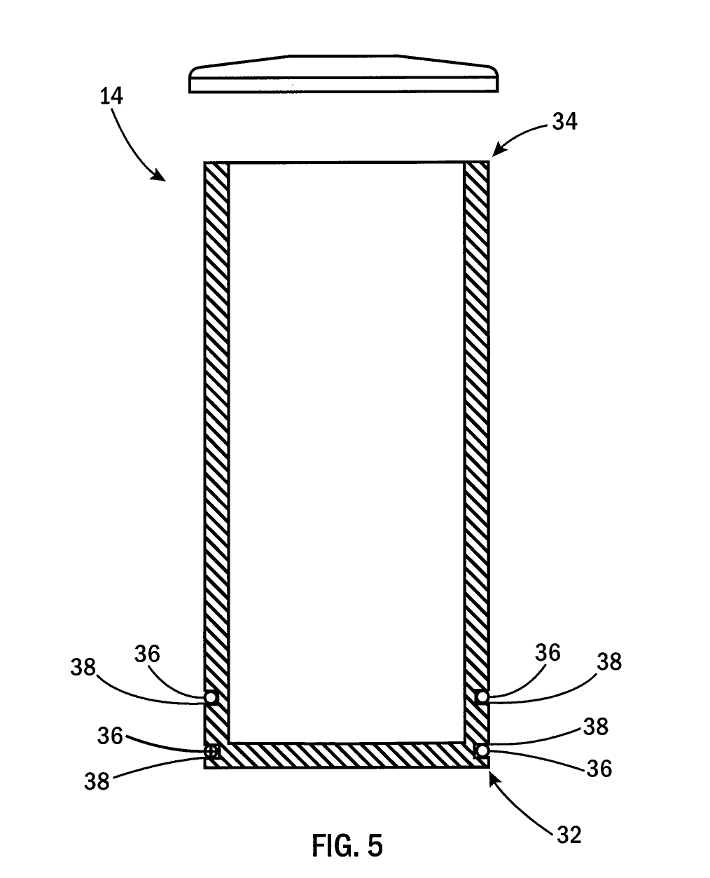

Referring now to FIG. 5 in particular, the plunger 14 according to the present invention is illustrated. Plunger 14 is sized and shaped so as to cooperate with the barrel 12 of device 10. In particular, plunger 14 comprises an outer diameter that is less than the inner diameter of the barrel 12. Understandably, plunger 14 may then be inserted into the interior cavity 16 of barrel 12 for expelling the cleansing solution stored within barrel 12 through outlet 24 as will be discussed herein. Plunger 14 is substantially hollow and includes a lower end 32 and an upper end 34 opposite lower end 34. Lower end 32 and upper end 34 are generally flat. Plunger 14 includes a pair of O-rings 36 circumferentially disposed therearound. O-rings 36 are preferably provided proximate lower end 34. Plunger 14 includes a pair of grooves 38 for receiving O-rings 36 and are sized and shaped to securely retain the O-rings 36. O-rings 36 are sized and shaped to form a seal between plunger 14 and barrel 12 such that the cleansing solution retained within barrel 12 is prevented from spilling out of the lower end of barrel 12 when displaced by plunger 14. That is, when the plunger 14 is inserted into barrel 12 to eject the cleansing solution from barrel 12 and onto the wound area, the cleansing solution housed within barrel 12 is maintained under pressure such that the cleansing solution is ejected from outlet 24 at a sufficient pressure.

The device 10 is preferably stored in a sealed package to maintain sterility and overall integrity. Once device 10 is removed from its packaging, the cleansing solution may be warmed if desired or clinically indicated as is generally understood in the art. To that end, device 10 may be provided with a temperature indicator (not shown) on its surface. As is generally understood, the temperature indicator is configured to visually provide the caregiver with a reading of the temperature of the cleansing solution such that the caregiver may warm or, alternatively, cool the cleansing solution by means generally understood in the art, such as a water bath. The temperature indicator preferably comprises at least two different colors for identifying the temperature of the cleansing solution. For instance, the temperature indicator may include a pale blue indicator for indicating that the temperature is approximately bath water temperature and a dark blue indicator for indicating that the temperature is greater than bath temperature, and thus the cleansing solution requires cooling prior to use. Further, a pink indicator may be provided for indicating an optimal temperature for aiding in vasoconstriction of the vessels in and around the wound area. Likewise, the pale blue indicator may be used to indicate a temperature at which vasodilatation will occur such that the bleeding of the wound will increase.

In operation, cap 28 is removed from tip 26 and the patient's wound is exposed. The caregiver then grasps barrel 12 and places the other hand on plunger 14 at end 34. Device 10 is preferably positioned approximately six inches away from the wound. Plunger 14 is inserted through bottom 20 of barrel 12 and compressed at a steady rate. The advancement of the plunger 14 is sufficient to burst seal 30 between the interior cavity 16 of barrel 12 and outlet 24 such that the cleansing solution is free to enter outlet 24 and be discharged through tip 26 and onto the wound. Barrel 12 is configured to hold a volume of cleansing solution such that the volume of cleansing solution in combination with the application of steady rate by the caregiver ensures that the cleansing solution is dispensed at the desired pressure, i.e., between approximately 8-11 psi. The cleansing solution is then applied to the entirety of the wound to ensure that all debris and foreign matter is removed. The wound area is then dried and the wound dressed to further protect it from infection. Thus, device 10 of the present invention provides a caregiver with a relatively simple wound irrigation device that eliminates or reduces user error with respect to the application of adequate pressure by providing a device capable of holding an adequate volume of cleansing solution such that the pressure of the discharged cleansing solution is sufficiently high.

1. A wound irrigation device comprising:
a barrel having a top end including a tip extending therefrom and a bottom end defining an aperture wherein the top end and the bottom end define an interior cavity for storing a predetermined volume of a cleansing solution therebetween;
a plunger sized and shaped to be selectively insertable into the barrel through the aperture for forcing the cleansing solution out of the tip under pressure, wherein the plunger comprises a substantially hollow structure and wherein at least one end of the plunger includes a substantially flat end for engagement with the cleansing solution.

2. The wound irrigation device of claim 1, wherein the top end of the barrel further includes a splash guard integrally formed therewith for protecting a caregiver from splashing of the discharged cleansing solution.

3. The wound irrigation device of claim 1, further comprising a selectively removable cap configured to cover the tip to maintain the sterility thereof.

4. The wound irrigation device of claim 1, wherein the tip comprises an outlet and a seal, wherein the seal is selectively burstable under pressure by the plunger to allow the stored volume of cleansing solution to enter the outlet so as to be discharged from the tip.

5. A method of irrigating a wound comprising the steps of:
providing a wound irrigation device comprising a barrel adapted to contain a predetermined volume of a cleansing solution and a plunger adapted for displacing a portion of the volume of cleansing solution;
positioning the wound irrigation device proximate a wound to be irrigated;
inserting the plunger into one end of the barrel;
compressing the predetermined volume of cleansing solution with the plunger such that the cleansing solution is discharged through an outlet of the barrel and into the wound to be irrigated.

6. The method of claim 5, further comprising the step of bringing the cleansing solution to a desired temperature.

7. The method of claim 6, wherein the desired temperature is configured to aid in one of vasodilation and vasoconstriction of vessels at or near the wound to be irrigated when the cleansing solution is applied.

8. The method of claim 6, wherein the step of bringing the cleansing solution to a desired temperature comprises the step of immersing the wound irrigation device in a water bath.
